(19) United States
(12) Patent Application Publication
(10) Pub. No.: US 2009/0004126 A1
(43) Pub. Date: Jan. 1, 2009

(54) Colour Compensating System

(76) Inventor: Linda Jane Lowndes, Queensland (AU)

Correspondence Address: CANTOR COLBURN, LLP, 20 Church Street, 22nd Floor, Hartford, CT, 06103, US

(21) Appl. No.: 11/658,940

(22) PCT Filed: Jul. 29, 2005
(86) PCT No.: PCT/AU2005/001119

§ 371 Date: Sep. 25, 2007

(30) Foreign Application Priority Data
Jul. 29, 2004 (AU) 2004904224
Jul. 29, 2004 (AU) 2004904225

(51) Int. Cl. A61K 8/00; A61Q 1/02; G01J 3/42

(52) U.S. Cl. 424/63; 427/2.31; 356/319

(57) ABSTRACT

A method for coating a skin blemish (10) such that the coated blemish visually blends into the surrounding skin (12) comprises measuring at least one colour property of the skin blemish (10) and measuring at least one colour property of the surrounding skin (12), suitably by using a spectrophotometer. From the measured properties, a coating composition having a compensatory colour is determined. When the skin blemish (10) is coated with the coating composition, the blemish (10) blends into the surrounding skin (12). The coating composition is not of identical colour to the surrounding skin (12) and the coating composition has a degree of translucency. The coated skin has a very natural look.

### FIELD OF THE INVENTION

The present invention generally relates to colour compensating systems and methods. More particularly, the present invention relates to methods for selecting a coating to obtain a desired appearance on a first surface, to a method for coating a surface to achieve a desired appearance on a first surface and to systems and apparatus for use in such methods.

### BACKGROUND OF THE INVENTION

Many individuals having skin blemishes that they may wish to conceal. For example, so-called port-wine birthmarks can appear on the face of many people. Such birthmarks are quite vivid in colour and can cause deep embarrassment and even psychological problems in people afflicted with such birthmarks. Significant skin blemishes can affect a person's confidence and self esteem, irrespective of whether they appear on the face or on other parts of the body.

Current treatment options for treating disfiguring skin blemishes involves either a cosmetic surgical procedure (such as laser treatment) to try to remove the blemish or using cosmetic preparations to cover or mask the blemish. Cosmetic surgery procedures can be painful, are expensive and have the risk of either failing to remove the blemish or causing unwanted side effects such as scarring or skin numbness. Accordingly, cosmetic surgical procedures are not always a viable treatment option.

Attempts to cover or mask the blemish using cosmetic compositions also cause difficulties. A first difficulty is often experienced in finding a cosmetic composition that can cover the blemish and match the surrounding skin colour. Furthermore, it is typically necessary to apply a thick layer of cosmetics over the blemish to successfully mask the blemish. This can cause an artificial, heavily made-up appearance which, in itself, can meet significant resistance. Thus, cosmetic covering of skin blemishes can also fail to achieve satisfactory outcomes. Automated colour matching has been practiced in a number of industries. For example, automated colour matching is used in the paint industry. In this application, a painted surface is measured using a calorimeter or spectrophotometer to determine one or more colour or visual properties of the painted surface. These measurements are then used to determine a paint colour that will match the painted surface. The selected paint can then be applied such that the newly painted surface matches the surrounding painted surface. Similar systems and methods can be used in areas such as colour testing of inks, colour control of paints and inks and matching of dental restoration work.

All of the above methods rely upon applying a coating, such as a paint, to a surface such that the coating fully masks the underlying surface. Efforts to utilise a similar principle to cover skin blemishes, i.e., selecting a cosmetic that is of identical colour to the skin surrounding the blemish and subsequently applying that cosmetic to the blemish to mask and cover the blemish, results in an unnatural appearance. This has led to resistance to using such a solution.

### SUMMARY OF THE INVENTION

In a first aspect, the present invention provides a method for selecting a coating to be applied to a first surface area such that the first surface area has at least one visual property that essentially matches at least one visual property of a second surface area, the method comprising the steps of:
i) measuring at least one visual property of the first surface area;
ii) measuring at least one visual property of the second surface area;
iii) using the measured at least one visual property of the first surface area and the measured at least one visual property of the second surface area to determine a coating having a compensatory visual property such that a coated surface of the first surface area has an essentially similar at least one visual property to the at least one visual property of the second surface area following application of the coating to the first surface area.

Preferably step (iii) comprises the steps of interrogating a database having information on the measured at least one visual property of the first surface area and information on the measured at least one visual property of the second surface area, said database further containing information on a required compensatory coating to be applied to the first surface area such that the coated first surface area has an essentially similar at least one visual property to the second surface area.

Preferably the database includes a matrix having information on the measured at least one visual property of the first surface area and information on the measured at least one visual property of the second surface area, said matrix further including information on the required compensatory coating.

In an alternative embodiment, step (iii) comprises the step of comparing the measured at least one visual property of the first surface area with the measured at least one visual property of the second surface area and determining the coating from the comparison. The comparison may comprise a subtraction involving the measured at least one visual property of the first and second surface areas to obtain a result and applying a correction factor to the result to determine the coating having a required visual property.

In a further embodiment, step (iii) comprises the step of applying a correctional formula utilizing the measured at least one visual property of the first surface and the at least one visual property of the second surface to determine a coating having the compensatory visual property.

Step (iii) may involve determining the compensatory visual property of the coating and providing a composition having the compensatory visual property. The step of providing the composition suitably comprises providing a list of ingredients and preparing the coating from the list of ingredients.

The steps of measuring at least one visual property of the first surface area and measuring at least one visual property of the second surface area preferably includes measuring at least one colour property of the first surface area and measuring at least one colour property of the second surface area. Even more preferably, the step of measuring at least one colour property of the first area involves measuring one or more of hue, chroma and lightness. The step of measuring at least one colour property of the second area preferably involves measuring one or more of hue, chroma and lightness. Preferably, two or more of hue, chroma and lightness are measured, more preferably all of hue, chroma and lightness are measured.

In another embodiment, the steps of measuring at least one visual property of the first surface area and measuring at least one visual property of the second surface area comprises measuring one or more of lightness, a red value and a yellow value. Preferably, each of lightness, the red value and the yellow value are measured in this embodiment.

The step of measuring the at least one visual property suitably is conducted by use of an appropriate instrument, such as a calorimeter or, more preferably, a spectrophotometer.

The coating used in the present invention has a compensatory visual property when applied to the first surface such that the first surface, when coated, exhibits an essentially similar visual property to the visual property of the second surface. The coating should not simply be an identical match to the second surface. Rather, the coating is such that the combination of the coating and the underlying first surface exhibits essentially the same visual property as the second surface. Rather than simply masking the first surface match with the second surface, the visual property of the first surface and the coating exhibit a combined or additive effect such that the coated first surface exhibits the desired visual property. To achieve this, the applied coating preferably has a degree of translucency, such that the underlying first surface is not merely masked by the coating. In other words, at the rate of application of the coating to the first surface area, the coating is not opaque.

In a second aspect, the present invention provides a method for coating a first surface area such that the coated first surface has at least one visual property that essentially matches at least one visual property of a second surface area, the method comprising the steps of:
i) measuring at least one visual property of the first surface area;
ii) measuring at least one visual property of the second surface area;
iii) using the measured at least one visual property of the first surface area and the measured at least one visual property of the second surface area to determine a coating having a compensatory visual property such that a coated surface of the first surface area has an essentially similar at least on visual property to the at least one visual property of the second surface area following application of the coating to the first surface area; and
iv) applying the determined coating to the first surface area.

Steps (i), (ii) and (iii) in preferred embodiments of the second aspect of the invention, correspond to preferred embodiments of the first aspect of the present invention.

In step (iv), the determined coating is applied to the first surface area. It is preferred that the coating is applied such that the coating does not simply mask the first surface area, but rather that the coated surface area exhibits a visual property that arises from an additive effect or combined effect between the underlying first surface area and the applied coating. Suitably, the coating has a degree of translucency in order to allow an additive or combined effect to be obtained from the underlying first surface area and the applied coating.

In a third aspect, the present invention provides a method for coating a skin blemish such that the coated blemish blends into the surrounding skin comprising:
i) measuring at least one colour property of the skin blemish;
ii) measuring at least one colour property of the surrounding skin;
iii) using the measured at least one colour property of the skin blemish and the at least one colour property of the surrounding skin to determine a cosmetic composition having a compensatory colour such that the skin blemish when coated with the cosmetic composition blends into the surrounding skin; and
iv) coating the skin blemish with the coating composition.

Preferably, the coating composition has a degree of translucency.

Preferably, the coating composition is not of identical colour to the surrounding skin.

The present invention also extends to systems and apparatus for use in the methods of the invention. In a fourth aspect, the present invention provides a system for selecting a coating to be applied to a first surface area such that the first surface area has at lest one visual property that essentially matches at least one visual property of a second surface area, the system including a measuring means for measuring the at least one visual property of the first surface area and the second surface area, and selection means for using the measured at least one visual property of the first surface area and the measured at least one visual property of the second surface area to select a coating having a compensatory visual property such that a coated surface of the first surface area has an essentially similar at least one visual property to the at least one visual property of the second surface area following application of the coating to the first surface area.

The selection means may comprise a computer database which includes information relating to the at least one visual property of the first surface area, to the at least one visual property of the second surface area, and to the compensatory visual property of the coating required to be applied to the first surface area such that the coated surface area has an essentially similar at least one visual property to the at least one visual property of the second surface area. The selection means may further comprise interrogation means for interrogating the computer database.

In another embodiment, the selection means may comprise calculation means utilizing the measured at least one visual property of the first surface area and the measured at least one visual property of the second surface area to calculate a compensatory at least one visual property of the coating required to achieve a coated surface on the first surface having an essentially similar at least one visual property to the second surface area. In this embodiment, determination of the required compensatory visual property for the coating is achieved by using a mathematical equation or a correlation involving the at least one visual property of the first surface area and the second surface area.

In yet another embodiment the selection means comprises determination means for determining the compensatory visual property required for the coating and coating selection means for selecting a coating having the compensatory visual property. The coating selection means may comprise a look-up table that cross-references the compensatory visual property of the coating composition.

The coating selection means may also provide or direct a user to a coating composition that may be prepared to obtain the desired compensatory visual property.

The measurement of the at least one visual property of the first surface and the second surface can also be used to determine which colour(s) is lacking in the surfaces so that it can be corrected or replaced.

The measuring means is preferably a colour measuring means. The measuring means may be a calorimeter or, more preferably, a spectrophotometer.

### BRIEF DESCRIPTION OF THE DRAWINGS

FIG. 1 is a diagrammatic representation of the arm of a patient having a skin blemish;

FIG. 2 is a plot of spectral reflectance vs wavelength for normal skin and the blemish; and

FIG. 3 is a plot of spectral reflectance vs wavelength for normal skin and the coated blemish.

### DETAILED DESCRIPTION OF THE EMBODIMENTS

The following description relates to preferred embodiments of the present invention.

The present invention is directed towards methods and apparatus for selecting a coating to apply to a first surface area such that the coated first surface area exhibits an essentially similar at least one visual property to a second surface area. In a preferred embodiment, the first surface area is a skin blemish on a person and the second surface area is an area of normal skin surrounding the skin blemish. The skin blemish may be any type of skin blemish, including a birthmark, a mole, a basal cell carcinoma, xeroderma pigmentosum, vitiligo, scars, burns, pigmentation, acne, veins, tattoos, bruises, etc. In this embodiment, the skin blemish is to be coated such that the skin blemish, after coating, looks essentially identical to the surrounding skin. The present inventor has found that previous attempts to cover skin blemishes by selecting an opaque coating that is exactly the same colour as the surrounding skin and subsequently applying that coating to the skin blemish to hide or mask the blemish resulted in an unnatural and overly-made up, almost plasticky, look. Thus, the present inventor has adopted a different approach. Rather than simply trying to hide the blemish, the present inventor applied a cosmetic coating that, in final appearance, has an additive or complementary effect with the underlying blemish such that the blemish) when coated, has a more natural appearance whilst still matching the appearance of the surrounding skin. Thus, the present invention does more than simply hide the blemish.

The coating composition (or cosmetic composition) used in the present invention preferably has a degree of translucency when applied to the skin. In addition to allowing for the additive effect with the underlying blemish to be obtained, a more natural appearance is also obtained.

Further details of the coating composition may be found in the applicant's co-pending Australian Provisional Patent Application No. 2004904224, filed 29 Jul. 2004 entitled “Skin Coating Composition and Uses Thereof”, and the International Patent Application claiming priority from Australia Patent Application No. 2004904224, the entire contents of which are herein incorporated by cross-reference.

The method of the present invention involves measuring at least one visual property of the first surface area and the second surface area. In the preferred embodiment, this involves taking measurements of the colour of the blemish and the surrounding skin. The colour measurements are preferably obtained using a spectrophotometer, such as those sold by X-Rite, Incorporated and sold in Australia by Applied Sensors Pty Ltd (trading as COLORITE Equipment).

In order to fully understand the preferred embodiment of the present invention, it will be necessary to briefly describe some methods of colour measurement. As is known to those skilled in the art, each colour has its own distinct appearance, based on three properties—hue, chroma and value. Value is also referred to as lightness. Each colour can be described and distinguished from other colours using those three properties.

Hue is generally perceived to be the “colour” of each colour, e.g., red, blue, green, yellow etc. Chroma describes the vividness or dullness of a colour. Chroma may be considered to describe how close a colour is to the pure hue or to grey. The luminous intensity of a colour, or its degree of lightness, is called its value. Colours can be classified as light or dark by comparing their values.

A number of scales also exist to measure colour. The earliest developed scale is the Munsell system of Colour Notation. This system assigns numerical values to the three properties of colours, namely hue, value and chroma. The Munsell scale was developed and based upon human perception of colour.

Modern day colour measurements utilise instruments, such as calorimeters and spectrophotometers, to measure the light. The instrument measures spectral data across the visible spectrum. After applying a correction for the illumination source used (typically by multiplying the measured spectral curve by the illumination source spectral curve), the resulting data is multiplied by a “standard observe”, which is a factor determined by the CIE (Commission Internationale de L′Eclairage—the International Commission on Illumination), to obtain tristimulus values X Y Z.

A more accurate colour measure can be obtained using chromacity coordinates Xyz. The coordinates Yxz, in which Y specifies the value or lightness determined by the tristimulus values, determine colour, in which xy are colour values in the chromacity diagram. For each value of Y, hue is represented at all points around the perimeter of the chromacity diagram. Chroma (or saturation) is represented by a movement and from the central white area out towards the diagrams perimeter, where 100% saturation equals pure hue.

CIE also recommends that alternate colour scales be used, namely CIELAB and CIELCH. These colour scales are based on the opponent—colour theory of colour vision, which states that two colours cannot be both green and red at the same time, nor blue and yellow at the same time. As a result, single values can be used to describe the red/green and yellow/blue attributes.

In CIELAB, a Cartesian space is used to denote lightness (L*), red/green (a*) and yellow/blue (b*). The Cartesian space in which L*, a* and b* are plotted is three dimensional. The point where a* and b* intersect at L*=O represent the hue and chroma of a colour. Lightness can then be added.

CIELCH is similar but uses polar coordinates to calculate a colour in a colour space. In CIELCH, L* denotes lightness, C* specifies chroma and h* denotes hue angle.

Comparisons of colour can also be made by calculating difference in their CIELAB or CIELCH values, and a total colour difference determined therefrom. Various tolerancing systems may also be used, including CIELAB tolerancing, CIELCH tolerancing, CMC tolerancing and CIE 94 tolerancing.

Other colour measurement systems use standards of whiteness or standards of yellowness. This is used particularly in the printing industry.

The above description of colour matching is based upon material kindly supplied by X-Rite, Incorporated.

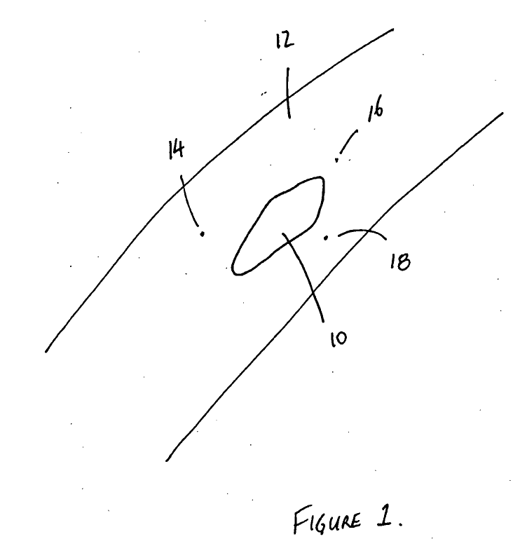

A preferred embodiment of the present invention will now be described with reference to the accompanying drawing. FIG. 1 shows a skin blemish 10 on a skin surface 12 of a patient. Skin blemish 10 is, in this instance, a port wine birthmark.

In order to select the appropriate coating composition to apply to skin blemish 10 such that the coated blemish matches the colour of normal skin 12 surrounding skin blemish 10 whilst obtaining a natural appearance, the method of the present invention takes one or more readings of colour from skin blemish 10. Preferably, a spectrophotometer is used to take the colour readings of skin blemish 10 and normal skin 12. Preferably, the colour readings taken from skin blemish 10 are taken from the darkest region of the skin blemish. Preferably, three readings are taken of the skin blemish.

The spectrophotometer is suitably used to take readings of hue, chroma and lightness or alternatively, the values of L*, a* and b* (lightness, red and yellow). If more than one colour reading is taken from skin blemish 10, it is preferred that the colour readings are averaged to obtain an average colour reading for skin blemish 10. The spectrophotometer is also used to take colour readings from the normal skin 12 surrounding skin blemish 10. Again, a single reading or a number of readings may be taken. For example, readings may be taken at three points 14, 16, 18 surrounding skin blemish 10. The colour readings for these three points may then be averaged to obtain an average colour reading for the normal skin 12.

Once the colour readings for skin blemish 10 and normal skin 12 have been obtained, using the spectrophotometer, a computer database is then interrogated. The computer database contains information relating to the colour properties of the blemish and the colour properties of the skin, as well as information relating to the appropriate colour properties of the coating composition to apply to the blemish such that the coated blemish exhibits the colour of the surrounding skin 12. The information relating to the colour properties of the coating to be applied to the blemish to obtain a desired appearance of the coated blemish has been obtained by the present inventor conducting experiments on her own skin and on trial patients to obtain the appropriate information for suitable look up tables. In other words, the information in the computer database presently has been obtained on an essentially empirical basis. However, it will also be appreciated that the appropriate information used to select the correct coating composition may be obtained by virtue of mathematical correlations obtained between the colour properties of the blemish, the colour properties of the skin and the compensatory colour properties of the coating composition.

The computer database may, in the above fashion, be used to select the coating composition. Alternatively, the computer database may provide information as to the desirable colour properties of the coating composition required to obtain the appropriate compensatory colour properties and this information may then be used to provide instructions to the user as to an appropriate coating composition to be prepared to obtain the desired compensatory colour properties. Such instructions may include a listing of ingredients to be mixed together to form the coating composition.

In another embodiment, a correctional formula may be developed from taking many empirical readings from skin, followed by analysis of the colour properties of the coating composition that achieves the final coated skin appearance. The correctional formula has been developed empirically.

Once the desired coating composition has been determined, that coating composition is either made up from its basic ingredients or selected from a group of coating compositions that have previously been made up and applied over the skin blemish. The coating composition may be applied by any means known to be suitable for applying cosmetic compositions to the skin. Examples include brushing, sponging, or airbrushing, with airbrushing being especially preferred.

The coating compositions that are used in the present invention suitably have a degree of translucency such that the colour of the coated skin blemish comprises the additive affects of the colour of the coating and the underlying colour of the blemish. Over-application of the coating composition should be avoided as this can result in an unnatural look being achieved.

The coating composition may be thought of as a simulated, flexible skin. The coating may be resistant to water and last for several days or longer on the skin before requiring re-coating. The coating may be removed prior to re-coating.

Further details of the computer database and of the coating composition may be found in the applicant's co-pending Australian Provisional Patent Application No. 2004904224, filed on 29 Jul. 2004 and the International Patent Application claiming priority therefrom entitled “Skin Coating Composition and Uses Thereof”, the entire contents of which are herein incorporated by cross-reference.

It is preferred that colour readings are taken on a recurring basis over a period of time or throughout the year so that the compensatory composition can be modified as appropriate to account for seasonal variations in skin colour (e.g. most people have darker coloured skin in summer than in winter) and any changes in the colour of the blemish.

In order to demonstrate the present invention, the normal skin and blemished skin of a patient was measured using a spectrophotometer. Three readings of each were taken and the lightness (L*), red (a*) and yellow (b*) values were as follows:

| | Normal Skin | Blemish |
|---|---|---|
| L* | 67.80 | 48.42 |
| a* | 8.11 | 12.09 |
| b* | 21.01 | 6.41 |

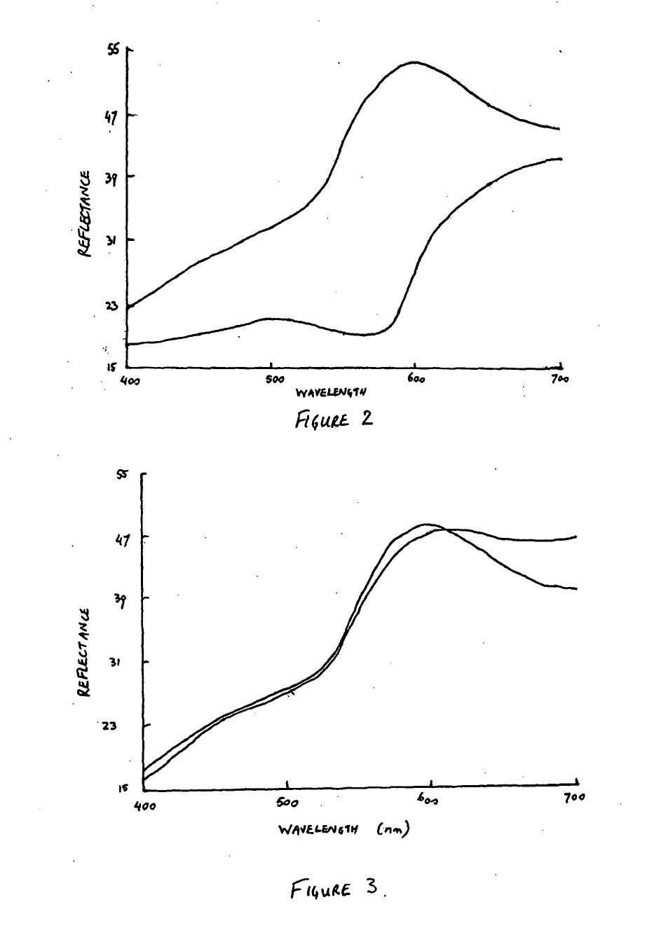

FIG. 2 shows a plot of reflectance vs wavelength for the normal skin and blemish. In FIG. 2, “standard” refers to the normal skin and “sample” refers to the blemish.

A corrective coating was then determined by the computer using a correlational or correctional formula. The following composition was recommended to be prepared as a result:

| Ingredient | wt % |
|---|---|
| Base | 88.0 |
| Black | 0.56 |
| Red | 0.57 |
| White | 7.23 |
| Yellow | 3.65 |

After mixing this formula, the composition was applied to the blemish by air-brushing. The coated blemish had a very natural look and blended in with the normal skin. Spectrophotometer readings were taken of the coated blemish and FIG. 3 shows a plot of reflectance vs wavelength for the normal skin (“standard” in FIG. 3) and the coated blemish (“sample” in FIG. 3). As can be seen from FIG. 3, very good correlation has been detained between the normal skin and the coated blemish. However, a visual comparison between the normal skin and a sample of the coating composition contained in a clear bottle showed that the coating composition by itself was of a significantly different colour to the normal skin.

It will be appreciated that the present invention may be susceptible to various modifications other than those specifically described. In particular, although spectrophotometers are particularly useful in the present invention, the invention also extends to cover use of any instrument that may be suitable for measuring visual properties or colour properties. Similarly, although the selection means preferably comprises a computer database or a computer program, the selection means can encompass any means by which the measured visual property of the first surface area and the measured visual property of the second surface area can be used to determine the appropriate coating having the required compensatory visual properties. For example, the selection means could be as simple as a graphical representation having the visual property of the first and second surface areas on respective X and Y axes and the selected composition being represented graphically on that graph, or the selection means may even comprise printed tables. The selection means may direct the user to a specific composition to select from pre-mixed or pre-manufactured compositions, or the selection means may direct the user to a recipe or composition mixture required to obtain the required compensatory visual properties.

Although preferred embodiments of the present invention take three readings from the patient's normal skin and the blemish, it will be appreciated that any number of readings may be taken.

The skin readings may also provide information on the colour lacking in the skin so that it can be corrected or replaced.

It will be understood that the invention disclosed and defined herein extends to all alternative combinations of two or more of the individual features mentioned or evident from the text or drawings. All of these different combinations constitute various alternative aspects of the invention.

The foregoing describes embodiments of the present invention and modifications, obvious to those skilled in the art can be made thereto, without departing from the scope of the present invention.

1. A method for selecting a coating to be applied to a first surface area such that the first surface area has at least one visual property that essentially matches at least one visual property of a second surface area, the method comprising the steps of:
a) measuring at least one visual property of the first surface area;
b) measuring at least one surface area of the second surface area; and
c) using the measured at least one visual property of the first surface area and the measured at least one visual property of the second surface area to determine a coating having a compensatory visual property such that a coated surface of the first surface area has an essentially similar at least one visual property to the at least one visual property of the second surface area following application of the coating to the first surface area.

2. A method as claimed in claim 1 wherein step (iii) comprises the steps of interrogating a database having information on the measured at least one visual property of the first surface area and information on the measured at least one visual property of the second surface area, said database further containing information on a required compensatory coating to be applied to the first surface area such that the coated first surface area has an essentially similar at least one visual property to the second surface area.

3. A method as claimed in claim 2 wherein the database includes a matrix having information on the measured at least one visual property of the first surface area and information on the measured at least one visual property of the second surface area, said matrix further including information on the required compensatory coating.

4. A method as claimed in claim 1 wherein step (iii) comprises the step of comparing the measured at least one visual property of the first surface area with the measured at least one visual property of the second surface area and determining the coating from the comparison.

5. A method as claimed in claim 4 wherein the comparison comprises a subtraction involving the measured at least one visual property of the first and second surface areas to obtain a result and applying a correction factor to the result to determine the coating having the required visual property.

6. A method as claimed in claim 1 wherein step (iii) comprises the step of applying a correctional formula utilizing the measured at least one visual property of the first surface and the at least one visual property of the second surface to determine a coating having the compensatory visual property.

7. A method as claimed in claim 1 wherein the steps of measuring at least one visual property of the first surface area and measuring at least one visual property of the second surface area comprise measuring at least one colour property of the first surface area and measuring at least one colour property of the second surface area.

8. A method as claimed in claim 7 wherein the steps of measuring at least one colour property of the first area and the second area involves measuring one or more of hue, chroma and lightness.

9. A method as claimed in claim 7 wherein the steps of measuring at least one visual property of the first surface area and measuring at least one visual property of the second surface area comprises measuring one or more of lightness, a red value and a yellow value.

10. A method as claimed in claim 7, wherein the step of measuring the at least one visual property is conducted by use of a colorimeter or a spectrophotometer.

11. A method as claimed in claim 1 wherein the coating has a degree of translucency when applied such that the underlying first surface is not merely masked by the coating and at the rate of application of the coating to the first surface area, the coating has a compensatory visual property when applied to the first surface such that the first surface, when coated, exhibits an essentially similar visual property to the visual property of the second surface.

12. A method as claimed in any claim 1 wherein the coating is not an identical match to the second surface.

13. A method for coating a first surface area such that the coated first surface has at least one visual property that essentially matches at least one visual property of a second surface area, the method comprising the steps of:
i) measuring at least one visual property of the first surface area;
ii) measuring at least one visual property of the second surface area;
iii) using the measured at least one visual property of the first surface area and the measured at least one visual property of the second surface area to determine a coating having a compensatory visual property such that a coated surface of the first surface area has an essentially similar at least on visual property to the at least one visual property of the second surface area following application of the coating to the first surface area; and
iv) applying the determined coating to the first surface area.

14. A method as claimed in claim 13 wherein in step (iv), the determined coating is applied to the first surface area such that the coating does not simply mask the first surface area, but rather that the coated surface area exhibits a visual property that arises from an additive effect or combined effect between the underlying first surface area and the applied coating.

15. A method for coating a skin blemish such that the coated blemish visually blends into the surrounding skin comprising:
i) measuring at least one colour property of the skin blemish;
ii) measuring at least one colour property of the surrounding skin;
iii) using the measured at least one colour property of the skin blemish and the at least one colour property of the surrounding skin to determine a coating composition having a compensatory colour such that the skin blemish when coated with the coating composition blends into the surrounding skin; and
iv) coating the skin blemish with the coating composition.

16. A method as claimed in claim 15 wherein the coating composition is not of identical colour to the surrounding skin and the coating composition has a degree of translucency.

17. A system for selecting a coating to be applied to a first surface area such that the first surface area has at least one visual property that essentially matches at least one visual property of a second surface area, the system including a measuring means for measuring the at least one visual property of the first surface area and the second surface area, and selection means for using the measured at least one visual property of the first surface area and the measured at least one visual property of the second surface area to select a coating having a compensatory visual property such that a coated surface of the first surface area has an essentially similar at least one visual property to the at least one visual property of the second surface area following application of the coating to the first surface area.

18. A system as claimed in claim 17 wherein the selection means comprises a computer database which includes information relating to the at least one visual property of the first surface area, to the at least one visual property of the second surface area, and to the compensatory visual property of the coating required to be applied to the first surface area such that the coated surface area has an essentially similar at least one visual property to the at least one visual property of the second surface area.

19. A system as claimed in claim 17 wherein the selection means comprises calculation means utilizing the measured at least one visual property of the first surface area and the measured at least one visual property of the second surface area to calculate a compensatory at least one visual property of the coating required to achieve a coated surface on the first surface having an essentially similar at least one visual property to the second surface area.

20. A system as claimed in claim 19 wherein the calculation means uses a mathematical equation or a correlation involving the at least one visual property of the first surface area and the second surface area.

21. A system as claimed in claim 17 wherein the selection means comprises determination means for determining the compensatory visual property required for the coating and coating selection means for selecting a coating having the compensatory visual property.

22. A system as claimed in claim 21 wherein the coating selection means comprises a look-up table that cross-references the compensatory visual property with coating composition.

23. A system as claimed in claim 17 wherein the coating selection means provides or directs a user to a coating composition for preparation to obtain the desired compensatory visual property.

24. A system as claimed in claim 17 wherein the measuring means is a colour measuring means selected from a colorimeter or a spectrophotometer.
